(19) United States
(12) Patent Application Publication
(10) Pub. No.: US 2019/0209749 A1
(43) Pub. Date: Jul. 11, 2019

(54) INSIDE BRA BREAST CUP BREAST PUMP

(71) Applicant: Natasha Meyers, Conroe, TX (US)

(72) Inventor: Natasha Meyers, Conroe, TX (US)

(21) Appl. No.: 15/864,260

(22) Filed: Jan. 8, 2018

(51) Int. Cl. A61M 1/06

(52) U.S. Cl. CPC A61M 1/064; A61M 2209/088; A61M 2209/10; A61M 2205/8206; A61M 2205/75

(57) ABSTRACT

The present invention is a breast pump having a concealed breast cup connected to both a pump hose and a milk hose, the pump hose and the milk hose connected to a pump, the pump has a milk container with a lid, a side bar control and a power button.

### BACKGROUND OF THE INVENTION

### Field of the Invention

This invention relates generally to human infant industry and more particularly to breast pumps.

### Description of the Related Art

The present invention is a breast pump that may have a breast cup, clamps, a pump hose, a milk hose, a milk container, a lid, a side bar control, a power button and a hip pack. The main purpose of the breast pump is to provide mothers a compact and automated breast pump. Many women utilize a breast pump to induce milk lactation, which is stored and later bottle fed to their child. Although breast pumps allow mothers to conveniently prepare nursing bottles before feeding hours or serve to help mothers alleviate full breasts, most machines are handheld and large this results in making milk collection a time consuming task that may also make some individuals feel uncomfortable while doing so on-the-go. Ingenious and practical, the present invention introduces a small electric breast pump that discreetly draws milk from inside a bra in order to facilitate the preparation process. Simple to use, one can attach the form-fitting suction cups of the present invention to each individual breast, underneath clothing, then press a power button found on the battery operating device to enable quick drainage. The suction cups will then effectively extract lactation which will travel through a hose into the collection container. The present invention is intended to be portable, designed with a pack that fastens to one's hip and houses the collection container as well as the side bar control buttons that permits users to adjust which suction cup is enabled in case one desires to pump a single breast, rather than both breasts simultaneously. The present invention can easily appeal to nursing mothers, as it provides an ideal alternative to traditional breast pumps, making it possible to be readily available at baby stores and online retailers.

The present invention is the only product of its kind to present an unobtrusive breast pump that does not require manual labor. The present invention is uniquely designed to be portable and electric, empowering women to comfortably extract breast milk in a discreet fashion without interrupting other daily tasks.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a front view of an inside bra breast cup, in accordance with one embodiment of the present invention.

FIG. 2 illustrates a front view of a pump, in accordance with one embodiment of the present invention.

FIG. 3 illustrates a front view of a milk collection container, in accordance with one embodiment of the present invention.

FIG. 4 illustrates a front view of a drying fabric, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

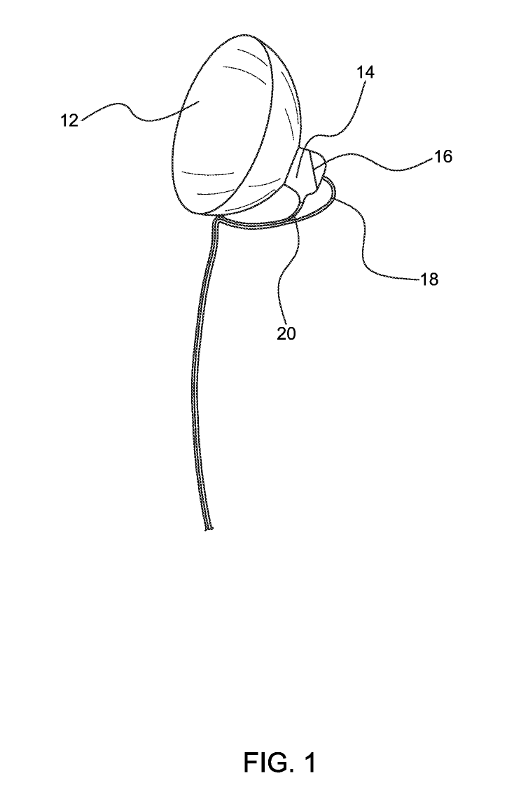

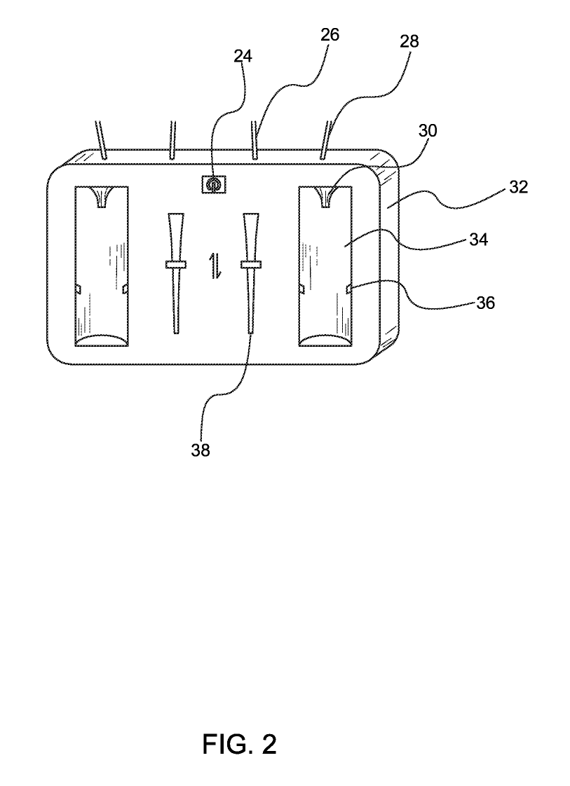

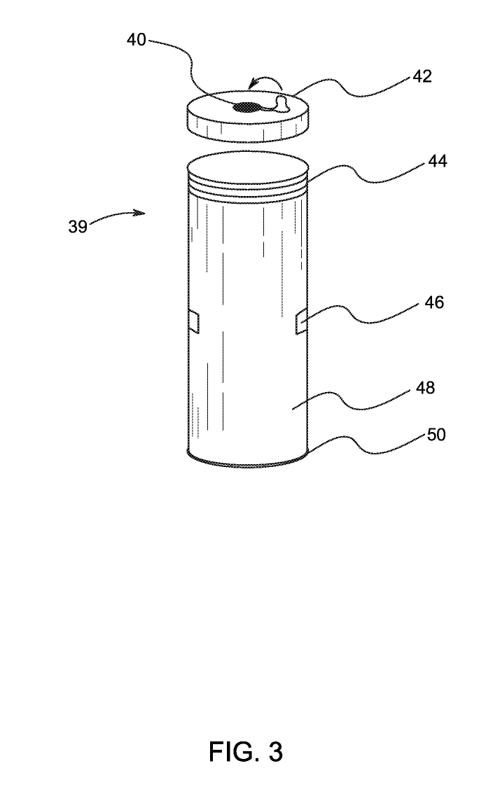

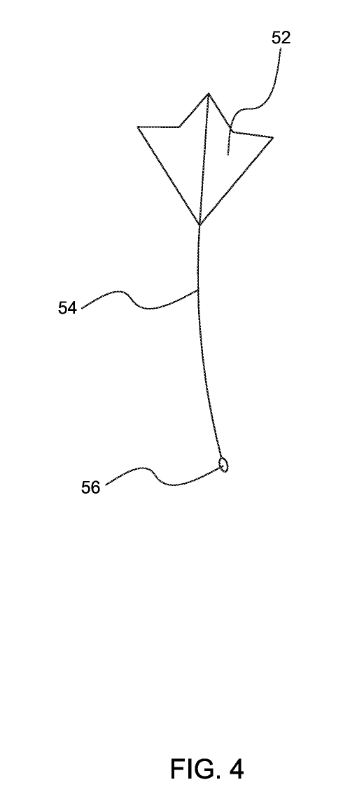

Referring now to FIG. 1, illustrated is a front view of the inside bra breast cup, in accordance with one embodiment of the present invention. In FIG. 1, illustrated is an inside bra breast cup 12, including a milk reservoir 14 and a screen 16. Shown are a pump hose 18 and a milk hose 20. The inside bra breast cup 12 may be made of soft flexible material and worn under a bra and may come in three different sizes. The milk reservoir 14 is designed for milk to drop into. The screen 16 keeps milk from going into the pump hose 18. FIG. 2, illustrated is a front view of the pump 32, in accordance with one embodiment of the present invention. In FIG. 2, illustrated is a power button 24. Shown are pump hose 26 and milk hose 28. Nozzle 30, collection container compartment 34 and clamps 36 are recessed into the pump. Shown is slide bar 38. Nozzle 30 is to drip milk into a collection container. Pump 32 may be battery operated and maybe attached to a user's hip. Slide bar 38 has a pump strength range for each breast independently. FIG. 3, illustrated is a front view of the milk collection container 39, in accordance with one embodiment of the present invention. In FIG. 3, illustrated is a hole 40 and a plug 42 both in a lid. Grooves 44 provide a screw feature for the lid. Indent 46 provides a slight indentation for clamps to hold the milk collection container 39 in place. Flat base 50 provides a stable bottom to place the milk collection container 39 in a refrigerator. Cylinder 48 allows a shape to be conveniently arranged in a bag or refrigerator. FIG. 4, illustrated is a front view of the drying fabric 52, in accordance with one embodiment of the present invention. In FIG. 4, illustrated is chain 54 and weight 56. Drying fabric 52 is to dry the inside of the milk hose 20. Chain 54 is used to pull the drying fabric 52 through the milk hose 20. Weight 56 fits inside the milk hose 20 to help pull the drying fabric 52 through milk hose 20.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

What is claimed is:

1. A breast pump comprising:
at least one breast cup adapted to be concealed inside a bra the breast cup connected to both a pump hose and a milk hose, the pump hose and the milk hose connected to a pump, the pump has a milk container with a lid, a side bar control and a power button.

2. The breast pump according to claim 1, wherein the pump is carried in a hip pack.

3. The breast pump according to claim 1, wherein at least one clamp holds the milk container to the pump.
